SEQUENCE LISTING (2 sequences)

SEQ ID NO: 1 - DNA, 24 bp, Homo sapiens
tagaggaggtgaggaagctgaagg

SEQ ID NO: 2 - DNA, 31 bp, Homo sapiens
tcatcggagatttccattttcctgttccatc

(19) United States
(12) Patent Application Publication
(10) Pub. No.: US 2015/0017190 A1
(43) Pub. Date: Jan. 15, 2015

(54) USE OF EPITOPES INDUCING SPECIFIC TOLERANCE FOR THE PREVENTION OF TISSUE REJECTION

(71) Applicants: Johann Bauer, Salzburg (AT); Monika Ettinger, Salzburg (AT); Iris Gratz, Salzburg (AT); Doris Schmid, Oberalm (AT)

(72) Inventors: Johann Bauer, Salzburg (AT); Monika Ettinger, Salzburg (AT); Iris Gratz, Salzburg (AT); Doris Schmid, Oberalm (AT)

(21) Appl. No.: 14/233,622

(22) PCT Filed: Jul. 26, 2012
(86) PCT No.: PCT/EP2012/064728

§ 371 Date: Jan. 17, 2014

(30) Foreign Application Priority Data
Jul. 26, 2011 (GB) 1112806.3

(51) Int. Cl. A61K 48/00; A61K 38/39

(52) U.S. Cl. CPC A61K 48/005; A61K 38/39; A61K 48/0075
USPC 424/184.1; 530/356; 536/23.5

(57) ABSTRACT

The present invention relates to a composition for use in the prevention of the rejection of skin tissue, comprising an effective amount of a peptide comprising an epitope of an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2) wherein said epitope induces immunological tolerance against its underlying polypeptide, and/or a nucleic acid for expressing a peptide comprising an epitope of said antigen as well as a gene therapy based on the composition, in the context of autoimmune blistering diseases, such as bullous pemphigoid or genetic skin diseases such as epidermolysisbullosa.

The present invention relates to a composition for use in the prevention of the rejection of skin tissue, comprising an effective amount of a peptide comprising an epitope of an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2) wherein said epitope induces immunological tolerance against its underlying polypeptide, and/or a nucleic acid for expressing a peptide comprising an epitope of said antigen as well as a gene therapy based on the composition, in the context of autoimmune blistering diseases, such as bullous pemphigoid, and genetic skin diseases, such as epidermolysis bullosa.

### BACKGROUND OF THE INVENTION

Immune responses to a therapeutic gene product are potentially serious complications in gene therapy. Due to the high immunogenicity of human bullous pemphigoid antigen 2 (hBPAG2), the induction and maintenance of tolerance towards this neo-antigen is critical for the success in the treatment of Epidermolysis bullosa(EB).

Using an animal model mimicking ex vivo gene therapy, Olasz and colleagues (Olasz et al., 2007) have shown that neo-expression of hBPAG2 lead to unwanted immune responses and rejection.

Xu et al (in: Xu L, Robinson N, Miller SD, Chan LS. Characterization of BALB/c mice B lymphocyte autoimmune responses to skin basement membrane component type XVII collagen, the target antigen of autoimmune skin disease bullous pemphigoid. Immunol. Lett. 2001 Jun. 1; 77(2):105-11) describe Bullous pemphigoid as an autoimmune blistering skin disease characterized by IgG autoantibodies targeting the skin basement membrane component type XVII collagen (BPAg2). To gain understanding of the disease's induction phase, Xu et al subcutaneously immunized adult BALB/c mice with peptides of human and/or the murine-equivalent BPAg2 pathogenic NC16A domain. Female mice were injected with peptides (human, murine, or combined human and murine), or PBS control emulsified in CFA, on a four-week interval. At the fourth and subsequent immunizations, all peptide-immunized mice were given murine peptides. Two weeks after the sixth immunization, ELISA detected IgG circulating autoantibodies against self-peptides in 92% (47/51) of mice immunized with murine peptides; whereas none of the preimmune sera, or the sera from PBS control-immunized mice reacted to the self-peptides.

The sequence of hBPAG2 (Protein) from Homo sapiens is published in the database as collagen, type XVII, alpha 1 at Acc. No. NP_{—}000494 (see also Sawamura, D., Li, K. H., Nomura, K., Sugita, Y., Christiano, A. M. and Uitto, J. Bullous pemphigoid antigen: cDNA cloning, cellular expression, and evidence for polymorphism of the human gene J. Invest. Dermatol. 96 (6), 908-915 (1991)). Bullous pemphigoidis an autoimmune skin disorder characterized by subepidermal blistering that results in large, tense bullae. IgG autoantibodies have been identified and are directed against 230 KD and 180 KD antigens, designated respectively as BP 230 Ag1 and BP 180 Ag2. BP 230 is on the intracellular hemidesmosome plaque and 180 BP is a transmembrane glycoprotein, whose extracellular domain reaches beyond the lamina lucida on the basement membrane zone, corresponding to filament anchorage (Pohla-Gubo G, Hintner H. Direct and indirect immunofluorescence for the diagnosis of bullous autoimmune diseases. Dermatol. Clin. 2011 July; 29 (3):365-72.).

Hirose et al. (in Hirose M, Recke A, Beckmann T, Shimizu A, Ishiko A, Bieber K, Westermann J, Zillikens D, Schmidt E, Ludwig R J. Repetitive Immunization Breaks Tolerance to Type XVII Collagen and Leads to Bullous Pemphigoid (BP) in Mice J Immunol. 2011 Jun. 24.) describe an experimental model inducing BP by immunization of immunocompetent mice with a recombinant form of the immunodominant 15th non-collagenous domain of murine BP180 (type XVII collagen). The homologous non-collagenous 16A domain of human BP180 has previously been identified as an immunodominant region in human BP. Immunization of female SJL/J mice with the murine peptide led to clinical disease within 14 wk in 56% of mice.

Significant progress has been made in corrective gene therapy of inherited skin diseases such as EB (Laimer et al., 2006; Mavilio et al., 2006; Murauer et al., 2011; Wally et al., 2008). However, in patients with null-mutations there is a high risk of rejection of gene therapy-treated tissue (Ghazizadeh et al., 2003). A particularly high risk of autoimmune responses exist in junctional EB missing laminin-5 or type XVII collagen and dystrophic EB lacking type VII collagen as these proteins are highly immunogenic (Sitaru et al., 2006; Spirito et al., 2001).

It is therefore an object of the present invention, to provide compositions and methods to help prevent and/or reduce the rejection of gene therapy-treated skin tissue expressing antigens, such as hBPAG2, in particular in the context of treating inherited skin diseases, such as EB and autoimmune bullous diseases.

In a first aspect of the present invention, this object is solved by a composition for use in the prevention of the rejection of skin tissue, comprising an effective amount of a peptide comprising an epitope of an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2) wherein said epitope induces immunological tolerance against its underlying polypeptide, and/or a nucleic acid for expressing a peptide comprising an epitope of said antigen, and wherein said epitope is not the full length polypeptide.

According to the present invention, preferred peptides comprising said epitope and which induce immunological tolerance have a length of between 6 and 400 amino acids, preferably between 10 and 200 amino acids, and most preferably comprise an extracellular domain of said polypeptide, such as, for example, NC16A, the immunodominant domain of hBPAG2. In these cases, the epitope is thus located in an extracellular presented peptide. The peptides comprising said epitope are preferably derived from the human polypeptide, such as human BPAG2, but can also be derived from mouse or other homologs of the polypeptides, such as BPAG2, as long as they include epitopes that are effective in inducing immunological tolerance.

In the context of the present invention, as a preferred example, NC16A, the immunodominant domain of hBPAG2, was used to in vivo-transfect skin of graft recipients prior to grafting to prevent immune reactions towards transplanted hBPAG2 expressing donor grafts. In contrast to control mice, 80% of wild-type mice gene gun transfected with NC16A showed indefinite (long-term) survival of skin grafts from mice expressing hBPAG2 in the epidermal basement membrane. Immunological tolerance was stable and transferable by lymphocytes of tolerant mice. CD25 depletion assays propose antigen specific regulatory T cells as potential mediators in the mechanism of tolerance induction. The inventors thus conclude that induction of these regulatory T cells is critical to the acceptance of transplanted ex vivo gene corrected skin. This is of relevance to patients undergoing gene therapy and has a potential impact on the treatment of autoimmune diseases.

“Immunological tolerance” in the context of the present invention shall mean the ability of the epitope (or epitopes) of an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2) as transfected/provided to induce a sufficient number of effector T cells and plasma cells as well as an antigen specific T_{reg} population that are suppressive enough to constrain effector mechanisms and in the subject as treated and to induce and/or maintain a stable tolerance against said antigen. “Immunological tolerance” can also be defined as a (full length) polypeptide-expressing graft survival of at least 75%, and preferably of at least 80%, over at least 100 days after transplantation into a subject.

Di Zenzo et al. (in: Di Zenzo G, Calabresi V, Olasz E B, Zambruno G, Yancey K B. Sequential intramolecular epitope spreading of humoral responses to human BPAG2 in a transgenic model. J Invest Dermatol. 2010 April; 130(4):1040-7. Epub 2009 Oct. 8.) describe Bullous pemphigoid (BP) as a subepidermal autoimmune disease characterized by a humoral response to an epidermal basement membrane (BM) component, BP antigen 2 (BPAG2). BP patients have IgG autoantibodies against an immunodominant BPAG2 extracellular domain termed NC 16A as well as additional epitopes located both in the intracellular and extracellular domains (ICD and ECD, respectively) of this autoantigen. To study the evolution of humoral responses to BPAG2, sequential serum samples obtained from C57BL/6Ncr mice grafted with otherwise syngeneic skin from transgenic mice expressing human BPAG2 (hBPAG2) in epidermal BM were studied for IgG reactivity to seven ECD and ICD hBPAG2 epitopes. All grafted mice developed specific IgG against hBPAG2 ECD and ICD epitopes. In seven of eight mice, anti-hBPAG2 IgG was initially directed against ECD epitopes; in six mice, humoral responses subsequently targeted additional ECD and ICD BPAG2 epitopes. In contrast to IgG specific for ECD epitopes, IgG against ICD epitopes was present at lower levels, detectable for shorter periods, and non-complement fixing. Interestingly, the appearance of IgG directed against ICD epitopes correlated with the development of graft loss in this experimental model. These studies provide a comprehensive and prospective characterization of the evolution of humoral immune responses to hBPAG2 in vivo.

In contrast to the state of the art, in the present invention the inventors have used the NC 16A domain of Col 17 coupled to DEC 205 in order to target the NC16 A domain to dendritic cells and induce tolerance. As shown in Sitaru et al, J Immunol 2006 (active mouse model of epidermolysis bullosa acquistita by application of COLT peptide fragments) and Hirose et al, J Immunology 2011 (active mouse model of bullous pemphigoid by application COL17 peptide fragments), the application of a peptide without addition of a tolerizing agent (peptide fragment) leads to an induction of autoimmunity rather than tolerization against this peptide. Therefore it was counterintuitive even for an expert in the field to see that application of the cDNA of the NC16 fragment leads to tolerance to the full length Col 17 molecule (without the addition of a further fragment).

WO 2008/114488A1 describes the use of a peptide fragment to bind to the autoantibodies occurring in bullous pemphigoid. Thereby, the autoantibody would not be able to bind to its target structure (Col 17) and thus a therapeutic effect would be achieved. This concept is called immunoabsorption (Herrero-Gonzalez et al; J Immunol 2006) and does not involve epidermal/dermal application of the cDNA of the antigen, as in the present case. Furthermore it does not involve specific immunoregulatory actions of T regulatory cells leading to tolerance. The only effect is to block the autoantibody binding to the antigen.

The composition according to the present invention are suitable for topical application, such as, for example, a pharmaceutically acceptable formulation, such as, for example, a gel, creme, paste, lotion, spray, suspension, dispersion salve, hydrogel or ointment formulation.

In another preferred aspect, the composition according to the present invention comprises a nucleic acid for expressing said peptide comprising an epitope of an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2). Respective expression constructs are well known in the state of the art and include, for example, “naked” DNA encoding said peptide comprising an epitope of an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2), as well as constructs including regulatory elements for expression, such as promoters for expression and/or sequences for integration into the chromosome of the skin cell to be transfected. Also preferred is a composition according to the present invention, wherein said composition is suitable for gene therapy, such as, for example, corrective gene therapy (i.e. correcting or “repairing” the molecular, histologic and functional abnormalities in the skin) and/or gene replacement therapy (i.e. introducing functional genes into the skin).

Most preferred is a composition according to the present invention, wherein said composition is suitable for gene gun transfer as, for example, described herein.

The present inventors have utilized gene gun treatment to transfect cells in the uppermost layers of the skin as an approach to replace specific genes absent in inherited genodermatoses. Gene gun delivery enables direct penetration of DNA coated gold particles through the cell membrane and subsequent expression of the antigen, followed by proteosomal degradation and presentation of antigenic peptides (Condon et al., 1996; Tang et al., 1992). Mature DCs have been shown to migrate to draining LN and are able to activate Ag specific CD4⁺ and CD8⁺ T cells leading to productive immune responses (Stoecklinger et al., 2007; Stoecklinger et al., 2011). Whereas the capacity to immunize is well appreciated, the potential of gene gun therapy to establish immune tolerance was yet unknown.

The present inventors, in a preferred example, used a gene gun approach to deliver NC16A, the immunodominant domain of hBPAG2, to induce tolerance to hBPAG2. Surprisingly, eighty percent of wild-type mice transfected with NC16A showed long-term survival of skin grafts expressing hBPAG2 (compared to null percent in control). Tolerance was stable and transferable, as hBPAG2-expressing grafts were maintained long-term and lymphocytes of tolerant mice could transfer tolerance to naïve hosts.

Gene gun transfection resulted in a dense Foxp3+ regulatory T cell infiltrate in grafts of tolerant mice and transient depletion of these cells resulted in a loss of tolerance induction. Taken together, the inventors thus could show that stable hBPAG2-specific tolerance is efficiently induced using gene gun delivery of NC16A through a regulatory T cell-dependent mechanism. This is of relevance to patients undergoing gene therapy and has also broader implications for the treatment of antigen-specific autoimmune diseases, as also tolerance to other polypeptides, such as an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, and a desmocollin can be achieved.

In another preferred aspect, the composition according to the present invention as described above is therefore particularly suitable for the treatment (in the context of) of an autoimmune blistering disease, such as pemphigus vulgaris, paraneoplastic pemphigus, bullous pemphigoid, cicatricial pemphigoid, dermatitis herpetiformis, linear IgA dermatosis, or epidermolysis bullosa acquisita.

A selection of autoimmune blistering diseases includes pemphigus vulgaris (PV), paraneoplastic pemphigus, bullous pemphigoid, cicatricial pemphigoid, dermatitis herpetiformis, linear IgA dermatosis and epidermolysis bullosa acquisita. Pemphigus encompasses a group of auto-immune blistering diseases of the skin and mucous membranes. Included in this group is pemphigus vulgaris, a bullous disease involving the skin and mucous membranes, which may be fatal if not treated with appropriate immunosuppressive agents. The detection of circulating antibodies against keratinocyte cell surface antigens led to the understanding that pemphigus was an autoimmune disease. These antigens comprise Desmoglein 1 and 3 as well as Plakoglobin.The blisters in PV result from the loss of cohesion of keratinocytes .

In some of these diseases the target antigen of autoimmunity is the same as the target gene of the genetic disease epidermolysis bullosa, e.g. in bullous pemphigoid and junctional EB: type XVII collagen; in epidermolysis bullosa acquisita and in dystrophic EB type: VII collagen. Therefore transfection of these and other antigens of the basement membrane zone during an ongoing autoimmune reaction might also be of therapeutic value.

Yet another preferred aspect then relates to a method for the prevention of the rejection of skin tissue expressing an antigen selected from the group of the polypeptides type XVII collagen, VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2), comprising administering to a subject in need of such prevention an effective amount of a composition according to the present invention as described herein. Preferably, said subject is a human.

Preferred is a method according to the present invention, wherein said prevention comprises gene therapy, such as, for example, corrective gene therapy and/or gene replacement therapy as described above. More preferred is a method according to the present invention, wherein said composition is administered using gene gun transfer as described above.

Also preferred is a method according to the present invention, wherein said method comprises the topical administration of the composition, such as, for example, as a gel, creme, paste, lotion, spray, suspension, dispersion salve, hydrogel or ointment formulation.

Yet another preferred aspect then relates to a method for the treatment of an autoimmune blistering disease, such as pemphigus vulgaris, paraneoplastic pemphigus, bullous pemphigoid, cicatricial pemphigoid, dermatitis herpetiformis, linear IgA dermatosis, epidermolysis bullosa acquista or, a genetic skin disease such as, for example, epidermolysis bullosa junctionalis, dystrophic epidermolysis bullosa comprising a method according to the present invention as above.

Immune responses towards a neo-antigen is a major limitation of gene therapy.The present inventors have thus investigated the potential of gene gun transfection in the induction of peripheral tolerance and have shown that this method of gene transfer can induce robust antigen-specific tolerance. Holcmann et al (Holcmann et al., 2009) proposed that in case of an expression of innocuous antigens in adult tissue continuously at low amounts, not even the presence of danger signals is sufficient to break tolerance. Since the inventors attempted to test the gene gun tolerance protocol in a setting comparable to an ex vivo gene therapy, they used the model of skin grafting .

Grafting of Wt C57BL/6 mice with hBPAG2 Tg skin leads to graft rejection as shown by Olasz et al. In accordance to Olasz et al, all mice that rejected the graft showed NC16A specific IgG, and the inventors additionally observed a high level of IgG in tolerant NC16A treated mice. Split skin stainings demonstrating the functional binding of BM in each group showed no difference between tolerant NC16A treated sera and sera from rejected negative controls. The inventors analyzed the NC16A specific IgG response in detail, revealing high serum levels of IgG1 and IgM antibodies, whereas the level of IgG2a and IgG2c was hardly detectable. But this low level was apparently sufficient to fix complement as shown by C3 fixation studies. In common autoimmune diseases such as Epidermolysis bullosa aquisita (EBA) and bullous systemic lupus erythematosus (BSLE), the presence of complement activating IgG subclasses of autoantibodies does not always correlate with disease activity (Gandhi et al., 2000), which is reminiscent of the presence of antibody titre without rejection in the system of the inventors. These studies indicate another mechanism involved in immune suppression that is not acting on the formation of anti-BM IgG antibodies.

As immune tolerance is an active process mediated by regulatory lymphocytes, gene gun transfection offers the possibility of inducing tolerance by inducing regulatory cell subsets as shown by Goudy et al and Lobell et al (Goudy et al., 2008; Lobell et al., 2003). Another example for a therapeutic gene gun approach was shown by Kageyama (Kageyama et al., 2004), who administered IL-4 by gene gun for the therapy of murine arthritis showing an immunosuppressive effect. Also, it has long been known that DNA vaccination can be suppressive in autoimmune diseases (Ruiz et al., 1999). Hence, in addition to other methods for gene transfer, gene gun transfection seems to be a particularly promising tool not only for immunization but also to induce immune suppression and tolerance.

Given the results of the inventors' antibody and lymphocyte transfer studies, the inventors conclude that antibody mediated mechanisms are not or at least not exclusively responsible for the observed effects of immune suppression, but lymphocytes are key mediators in mediating graft acceptance and tolerance induction. As transplantation tolerance could only be transferred by lymphocytes from tolerant grafted mice and not from gene gun transfected mice, it seems likely that the population of NC 16A-specific T_{reg} cells further expands over time and/or due to the Tg graft placement (Kataoka et al., 2002; Qin et al., 1993).

T_{regs} show a kinetic—in the first 3 weeks, T_{regs} are at site of graft in tolerant mice, maybe suppressing the mast effector cells and therefore inhibiting graft rejection. According to the inventors' hypothesis, some T_{regs} are staying at site of graft presumably regulating and suppressing T effector cells, but the majority is migrating as induced T_{regs} in the periphery to the draining lymph nodes to suppress Teff. In rejecting mice, T_{regs} are later on after acute rejection (d26) at site of graft to constrain the immune response.

In vivo depletion of T_{reg} cells at day 42 using an antibody to CD25 ablated the graft protecting effect in 50% of cases. Thus, the inventors suppose that in 50% of cases T effector cells are released from suppression indicating that NC 16A-specific T_{reg} cells are induced by gene gun transfection playing a major role in graft acceptance. Furthermore, the inventors speculate that the remaining 50% of mice maintaining the graft lack a Teff/memory cell population. One could hypothesize that deletion of Teff/memory is another mechanism of tolerance in this system. Deletion could be not 100%, being causative for the variance. As CD4⁺CD25⁺ T cells could transfer tolerance the inventors attribute T_{reg} cells as major executives in inducing grafting tolerance.

In summary, gene gun treatment and grafting very efficiently induced effector T cells and plasma cells as well as an antigen specific T_{reg} population. These T_{regs} are suppressive enough to constrain effector mechanisms and seem to be the major cell type in inducing tolerance and potentially also in the maintenance of tolerance. Deletion of antigen specific effector T cells by undergoing activation induced cell death (AICD) is one option to stabilize tolerance (von Herrath and Harrison, 2003).

In conclusion, the inventors have demonstrated the prevention of neo-antigen mediated rejection of skin grafts by in vivo gene gun transfection with NC16A. The potential of this method in a clinical setting of ex vivo gene therapy is obvious in case of a treatment of genetic skin disease such as epidermolysis bullosa junctionalis, dystrophic epidermolysis. Also an ongoing autoimmune reaction as in the case of autoimmune blistering diseases, such as pemphigus and bullous pemphigoid might be a possible application of this approach.

The present invention shall now be further described in the following examples, nevertheless, without being limited thereto. For the purposes of the present invention, all references as cited are incorporated by reference in their entireties. The accompanying Figures show:

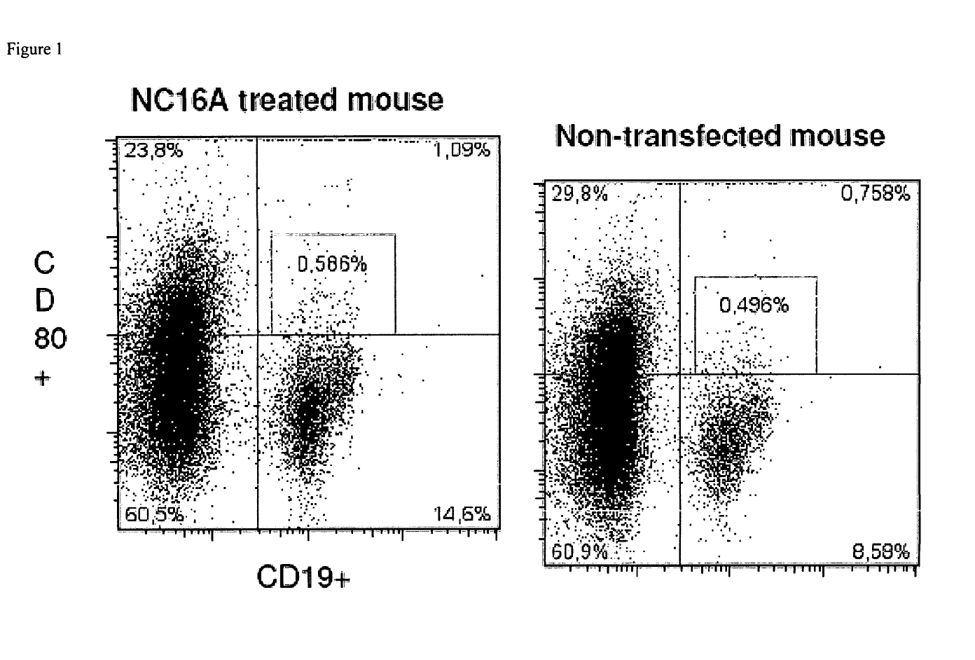

FIG. 1: Gene gun transfection of C57BL/6 mice does not elicit B cell activation. Lymph nodes of gene gun transfected C57BL/6 mice were taken for FACS analysis on day 28 post transfection. B cells/CD 19⁺ cells from NC16A treated mice showed activation levels comparable to non-transfected mice (pool of mice n=5).

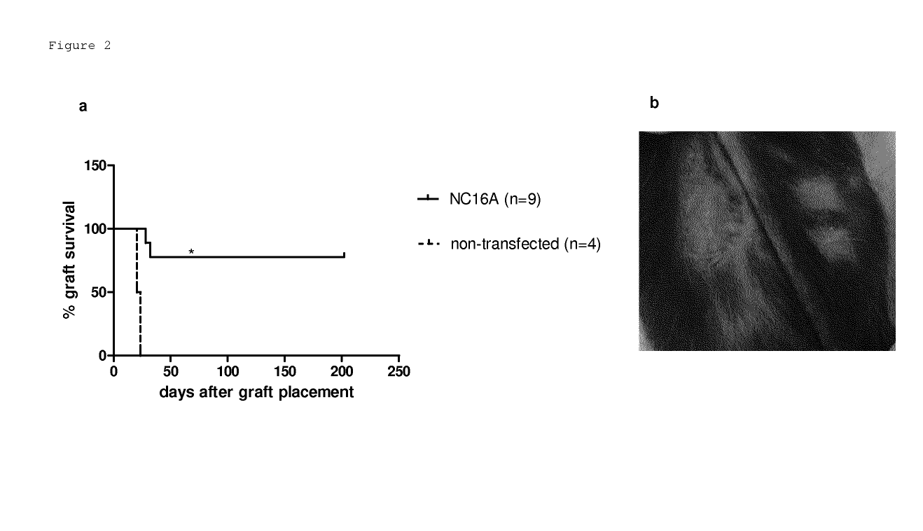

FIG. 2: a) Gene gun transfection with NC16A prevents graft rejection. Skin of heterozygous hBPAG2 Tg mice was grafted onto syngeneic Wt C57BL/6 recipients. Recipients were gene gun transfected with NC16A prior to grafting, the control group remained untreated. 7 of 9 NC16A treated mice accepted the graft, whereas all animals of the negative control group rejected the graft (observation period 98 days). A second graft was placed on the opposite site on day 64 (*) of NC16A treated mice, showing continuous graft survival (full observation period 202 days) (n=2)b) Graft appearance of the two accepted skin grafts. First graft was placed on the left side (202 days), second graft on the right side (138 days).

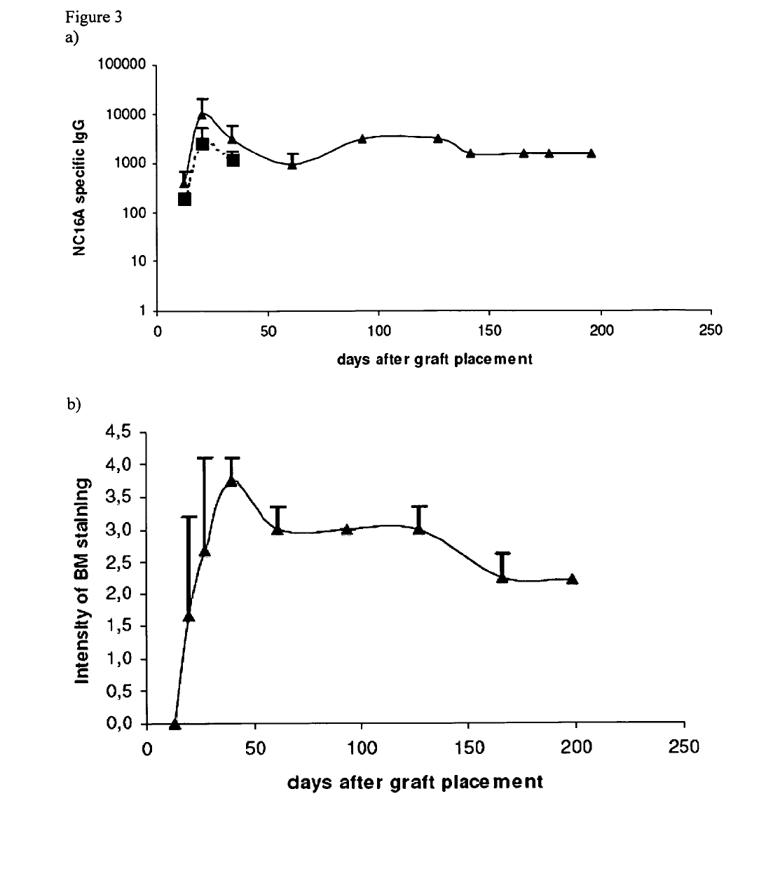

FIG. 3: Gene gun treatment did not prevent anti-BM IgG production following hBPAG2 Tg skin graft placement. A) NC16A specific IgG ELISA, B) anti-BM IgG on split skin. Anti BM IgG developed in all NC16A treated and grafted mice in similar way as in non-transfected grafted mice. anti-BM IgG titres arose at day 20 and were durable and stable over the observation period (98 days). anti-BM IgG titres did not remarkably change after placement of the second graft (n=2) and remained high until day 202 (NC16A treated mice are displayed as triangles, non-transfected mice as squares).

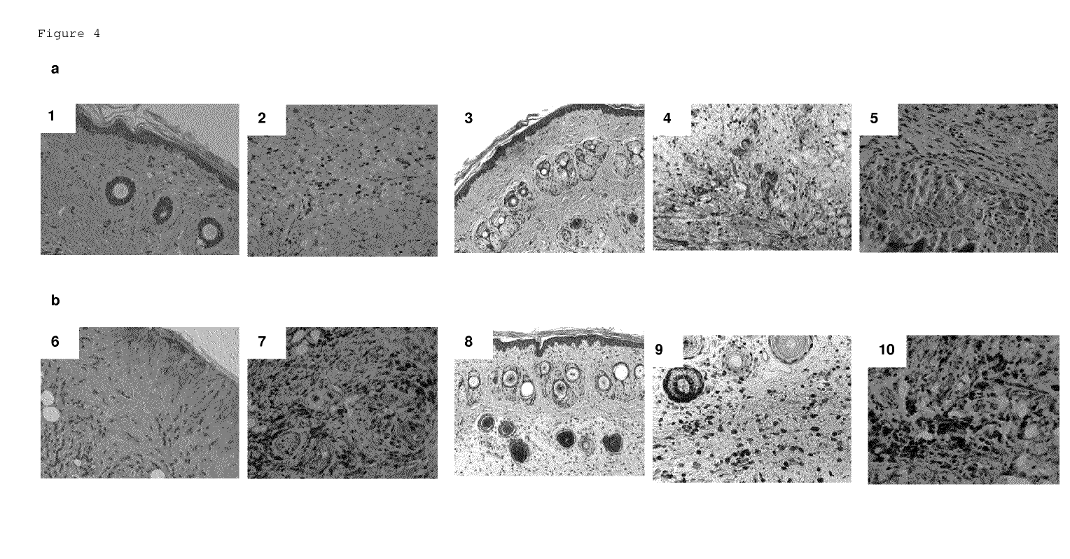

FIG. 4: Increased T_{regs} and lack of mast cells in tolerance grafts. a NC16A gene gun transfected and grafted mouse, b non-transfected grafted mouse. 1+6 HE staining (40×), 2+7 c-kit (mast cell staining, dark brown) (40×). Graft rejection correlated positively with grade of inflammation and infiltration of mast cells. T_{regs} are situated in tolerant grafts until day 18 and migrate into the periphery of lymphatics on day 26.3+8 HE staining (40×), 4+9 Foxp3 staining (dark brown) (40×) day 18, 5+10 Foxp3 staining day 26 (dark brown) (40×). Representative examples out of 2 biopsies.

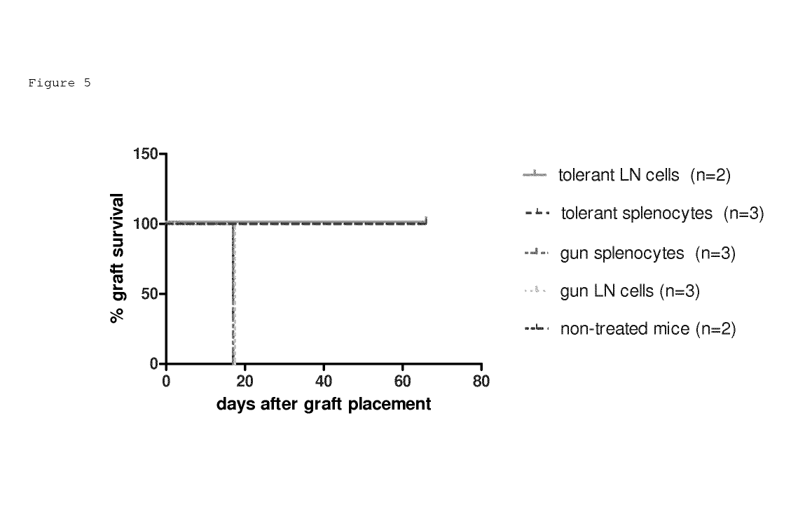

FIG. 5: Tolerance to hBPAG2 Tg skin can be transferred by lymphocytes from tolerant mice. Splenocytes or lymph node cells from tolerant mice (day 98) were injected to wt mice one day prior to grafting hBPAG2-expressing skin (n=11). Tg skin grafts were lost in mice which received lymphocytes from gene gun treated mice. Non-treated mice served as control.

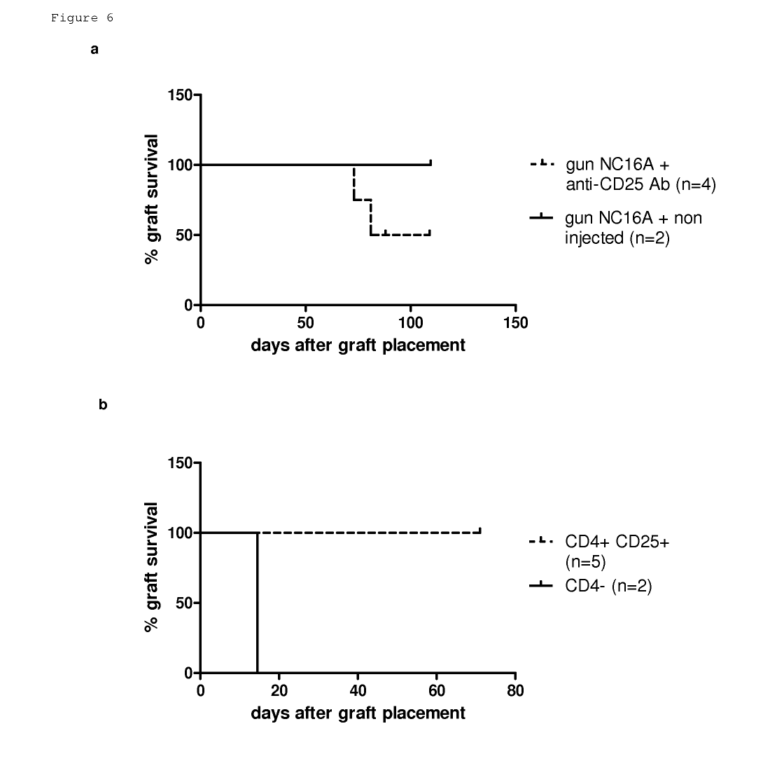

FIG. 6: a) T_{regs} play major role in tolerance induction. Wt mice were gene gun transfected with NC16A prior to grafting and injected with anti-CD25 antibody at day 42. 2 of 4 CD25⁺ depleted mice rejected the graft within week 5 and 6. b) CD4⁺CD25⁺ cells are responsible for graft acceptance. Wt mice were injected with CD4⁺CD25⁺ (1×10⁶ per mouse) and CD4⁺ T cells (5×10⁷ per mouse) of tolerant mice bearing the graft for 96 days, one day before grafting. Injection of CD4⁺CD25⁺ cells inhibited graft rejection.

### EXAMPLES

Materials and methods

Mice. C57BL/6 hBPAG2 tg (Tg) mice, expressing hBPAG2 in the basal membrane (BM) of the skin (hBPAG2 Tg) were obtained by Kim Yancey, Southwestern Medical School at Dallas, Tex., USA). Female BALB/c and C57BL/6 mice (8-10 wk old) were purchased from Charles River Laboratories (Sulzfeld, Germany). Mice were housed under SPF conditions in the animal facility at the University of Salzburg and at the Paracelsus Medical University Salzburg, Austria according to the institutional and national guidelines for animal care and use. All experiments described in this study were approved by the Austrian Federal Ministry for Science and Research Austria.

Design of construct. NC16A was amplified from cDNA of human keratinocytes using primers specific for NC16A (Genebank accession number NM_{—}000494, primer sequence fwd: 5′ tagaggaggtgaggaagctgaagg 3′ (SEQ ID No. 1), rev: 5′ tcatcggagatttccattttcctgttccatc 3′ (SEQ ID No. 2) inserting a stop codon). The inventors truncated NC16A (10 amino acids) at the 5′ end to delete the coiled coil motif located on its N terminus. Constructs were cloned into pEF6/V5 His TOPO vector (Invitrogen, Karlsruhe, Germany) including the ER-targeting sequence of human tissue plasminogen activator (TPA leader) for secretion of the constructs and 6× His-tag for detection. Constructs were purified by use of the Qiagen Endo free kit (Qiagen, Hilden, Germany).

Epidermal transfection using gene gun. The skin of BALB/c and C57BL/6 mice was transfected in vivo with constructs using gene gun transfection. Preparation of DNA coated-gold particles was performed according to manufacturer's instructions. Briefly, plasmid DNA was precipitated onto gold beads (1.6 μm diameter, Bio-Rad, Munich, Germany) by CaCl₂ in the presence of spermidine (Sigma-Aldrich, Vienna, Austria). Mice received two non-overlapping shots onto the shaved abdominal skin. With each shot, 1 μg of DNA immobilized onto 0.5mg gold particles was delivered at a pressure of 400 pounds per square inch (psi) with a Helios gene gun (Bio-Rad).

Transient in vitro transfection studies. NIH 3T3 cells were obtained from ATCC (Manassas, Va.) and cultured in HyClone® DMEM High Glucose (containing 4500 mg/L Glucose, 4 mM L-Glutamine, 10% FCIII, w/o sodium pyruvate; Thermo Fisher Scientific Inc., Vienna, Austria). Cells were transfected using the lipofectamin Eco-Transfect™, according to the instruction manual (Protocol Eco-transfect™, OZ Biosciences, Marseille, France). To assess protein expression, crude cell lysates of transfected NIH 3T3 were prepared after 48 hours and resolved on 10% SDS-PAGE under reducing conditions. After blotting onto nitrocellulose (Amersham, Buckinghampshire, UK) by standard techniques, the blots were blocked with blocking buffer and then incubated with anti-his-HRP conjugate solution (Qiagen) according to QlAexpress ® anti-his HRP Conjugate manufacturers protocol (Qiagen). Bands were visualized with Immun-Star Western C Kit (Bio-Rad).

Flow cytometric analysis. LN cells were isolated from mechanically disrupted LN tissue by collagenase D (1 mg/ml in HBSS) and DNase I 0.12 mg/ml in HBSS digestion (Roche; Vienna, Austria, 40 min at37° C., with gentle agitation) and filtered through 70-μmnylon mesh filters. For surface staining, cells were incubated with anti-CD4 mAb, anti-CD8 mAb, anti-CD19 mAb and anti-CD11c mAb (eBioscience) in combination with various activation markers (CD86, CD62L, CD69 and CD25, all from eBioscience). Cells were recorded on a Beckman Coulter flow cytometer (FC500) and analyzed by FlowJo (Tree Star, Inc. Ashland, Oreg., US)

Serum preparation and ELISA. Serum was prepared weekly from blood samples. For detection of NC16A specific immunoglobulins, high protein-binding ELISA plates (NuncMaxiSorp®, eBioscience) were coated overnight at 4° C. with 1 μg/ml recombinant NC16A in PBS. Plates were blocked (PBS containing 0.1% Tween and 0.5% BSA) for 1 hour at room temperature (RT). Serial dilutions of mouse sera were incubated 1 hour at RT. Plates were washed and Ag-specific Ig were detected with isotype specific HRP-conjugated detection antibodies (IgG-, IgG2a-, IgM-, IgE -HRP respectively, Serotec, Dusseldorf, Germany). Antibody titres were determined by end point titration and expressed as the dilution factor yielding a signal higher than three times the quantification limit.

Indirect immunofluorescence microscopy. Sera from mice grafted with hBPAG2 Tg skin were diluted 1:300 and tested for anti-basement membrane (BM) IgG by indirect immunofluorescence microscopy of 1M NaCl split human skin . Immunofluorescence stainings were examined by one independent observer and scored as follows: negative (-), weak (+), moderate (++), strong (+++), very strong (++++). This scoring was based on the intensity of BM staining according to routine immunofluorescence microscopy in bullous autoimmune diseases. ++++ corresponds to a titre of 1:10 000 in ELISA.

Skin grafting. Tail skin was taken from hBPAG2 Tg mice and grafted onto the backs of gender-matched Wt C57BL/6 recipients (Rosenberg and Singer, 1988). Bandages were removed at day 7 and grafts were checked twice a week until day 30, then weekly, for viability and size Skin grafts were graded as lost if their area became 70% or less of their original size.

Histological analysis of skin grafts. 4 mm biopsies were obtained at various time points after grafting. Samples were placed into Michel's medium, embedded in paraffin, sectioned at 5 μm thickness and stained with Hematoxylin and Eosin. For immunohistochemistry, sections were deparaffinized through xylene and graded alcohols, followed by a heat induced antigen retrieval in EDTA buffer pH 9.0 at 98° C. for 40 minutes. Validated polymer-based (Envision™, Dako DN) immunohistochemistry was performed in combination with a peroxidase substrate-chromogen system. Following primary antibodies were used: for T_{regs} staining rabbit polyclonal to Foxp3 antibody (ab54501 abcam, Cambridge, UK, 1:50), for mast cell staining anti-mouse CD117/cKit (ab5506 abcam, 1:50).

Adoptive transfer studies. A total of 5×10⁷ splenocytes and 1×10⁷ cells from LN of NC16A treated Wt mice bearing intact and viable hBPAG2 Tg skin grafts for 98 days, were adoptively transferred to Wt mice one day before grafting. Mice treated with NC16A (2 weeks after gene gun) served as lymphocyte donors in a separate set of experiments. As control served non-treated mice. Furthermore, CD4⁺CD25⁺T cells were isolated from splenocytes and LN cells from mice bearing intact and viable hBPAG2 Tg skin grafts for 98 days, using CD4⁺CD25⁺ Regulatory T Cell Isolation Kit (MACS, MiltenyiBiotec, BergischGladbach, Germany). CD4⁺CD25⁺ (1×10⁶ per mouse) and CD4⁺(5×10⁷ per mouse) cells were injected into Wt mice one day prior to grafting. Graft viability was observed weekly.

CD25⁺ T cell depletion studies. To examine if T_{reg} depletion abolishes the maintenance of tolerance, mice were treated with gene gun prior to grafting and injected i.v. with 50 μg anti-mouse CD25 antibody (Clone PC61.5, eBioscience) at day 42.

### Results

Gene gun transfection of NC16A is non-immunogenic. To test the correct expression of TPA-NC16A, the inventors performed in vitro transfection studies followed by Western Blot analysis. NC16A showed expression at correct size. To investigate if in vivo skin transfection elicits an antibody response against NC16A, using the gene gun system the inventors transfected C57BL/6 mice with NC16A. Sera were collected before transfection and weekly afterwards. Using ELISA specific for IgG against NC16A as well as indirect immune fluorescence microscopy on human split skin, NC16A-specific antibody was not detected at any time point after a single or multiple transfections. Furthermore, lymph nodes of gene gun treated C57BL/6 mice were taken for ELISPOT analysis and FACS analysis on day 11 (n=3) and day 32 (n=2). All groups of mice showed B cell activation levels comparable to naïve mice (FIG. 1). To test whether lack of an antibody response was due to a failure of antigen-expression or incorrect protein folding, the inventors performed gene gun transfection in BALB/c mice as gene gun transfection of this strain has been reported to elicit strong humoral immune responses. Transfection with NC16A into BALB/c skin did not result in NC16A-specific IgG production.

Gene gun delivery of NC16A prevents graft rejection and induces stable tolerance. Given that gene gun delivery of NC16A did not result in a productive humoral immune response, the inventors hypothesized that this method of gene delivery could induce tolerance. To test this hypothesis, the inventors utilized a skin grafting approach whereby syngeneic hBPAG2-expressing skin was grafted onto mice that were gene gun transfected with NC16A prior to grafting. To determine if transfection with NC16A leads to graft survival in Wt mice, full thickness 1 cm² grafts from the tails of Tg mice (heterozygous hBPAG2 Tg) were placed onto the flanks of gender-matched, syngeneic recipients. Eighty percent of NC16A transfected mice retained hBPAG2 Tg skin grafts (n=7/9) for >98 days whereas all negative controls (i.e. non-transfected Wt animals) rejected grafts within 28 days. To assess the durability of tolerance induced in NC16A-treated mice, a second graft was placed on mice that had retained an initial Tg skin graft for >60 days (n=2). 2 of 2 mice in this series accepted both the initial and second Tg skin grafts for the full observation period (>200 days) (FIG. 2). In order to exclude the possibility that transgene silencing in the skin graft was a mechanism of graft acceptance, the inventors verified transgene expression in grafted skin by IF staining using an antibody against hBPAG2 showing long-term expression.

Gene gun treatment does not prevent anti-BM IgG production following hBPAG2 Tg skin graft placement. All mice grafted with gender-matched, syngeneic hBPAG2 Tg skin developed NC16A specific IgG with high titres (1,000-10,000) within the first 20 days post first graft placement independent of way of treatment (observation period 98 days) (FIG. 3a). Similar kinetics was observed using indirect immunofluorescence microscopy, revealing that Ab bind human epidermal BM (FIG. 3b). Titres remained at these levels long-term (98 days). The NC16A treated group showed no increase in titre after placement of the second graft—the titre remained high over 202 days (3,200, post first grafting). Anti-BM IgG detected in immunofluorescence analysis of human split skin corresponded with NC16A specific IgG levels in ELISA. Furthermore, analysis of Ig subtypes revealed high IgG1, IgM and IgE (titer 4,000-30,000) but very low IgG2a (just over background) over time. Complement binding ability of the antibodies was examined by conducting C3 fixation assays using the split skin protocol. C3 staining was observed in rejected skin of naïve mice, as well as in accepted grafts of NC16A treated mice.

Tolerant grafts lack inflammation. To characterize the inflammatory response in skin grafts from NC16A-transfected tolerant mice and non-tolerant control mice, skin sections were harvested at days 12, 18, 28, 64 and 202 post grafting and tissue samples were subjected to histological examinations. 12 and 18 days after grafting, an inflammatory infiltrate in the dermis comprising numerous/abundant mast cells was detected. At day 28, sites of graft rejection showed cutaneous ulceration as well as fibrosis and a dense mast cell infiltrate in the dermis. 64 days after grafting, grafted skin was completed rejected and the wound was fully healed. In contrast, biopsies of grafts from NC16A-transfected tolerant mice displayed a spotty parakeratotic epidermis with subtle superficial dermal fibrosis and a lack of a mast cell infiltration (FIG. 4).

Lymphocytes from NC16A-transfected mice can adoptively transfer BPAG2-specific tolerance. In an attempt to elucidate whether lymphocytes play a role the maintenance of tolerance in NC16A-transfected mice, the inventors injected wt mice with splenocytes or lymph node cells of tolerant mice (n=11) one day prior to grafting hBPAG2-expressing skin. Tolerant mice which accepted the graft after gene gun transfection for 98 days as well as mice which were treated with gene gun two weeks before cell isolation served as donors. Both splenocytes (n=3/3) and lymph node cells (n=2/2) of tolerant mice resulted in long-term graft acceptance, in contrast to those which received cells from mice gene gunned without grafting (FIG. 5).

Regulatory T cells play a major role in the establishment and maintenance of tolerance in NC16A-transfected mice. Given that T_{reg} cells have been shown to play a major role in the maintenance of peripheral tolerance (Bala and Moudgil, 2006; Joffre et al., 2008) the inventors tested whether these cells were important in the inventors' model of gene-induced tolerance. Immunohistological analysis of accepted skin grafts stained for Foxp3⁺ cells showed a deep dermal T_{regs} infiltrate at the site of the graft on day 14 and 18, decreasing on day 26. Contrary, non-transfected mice showed a T_{regs} infiltrate on day 18, increasing on day 28 at time of rejection. Furthermore, this T_{regs} infiltrate is situated in the upper dermis and the area of rejected skin (FIG. 4). To further examine the role of T_{regs} in tolerance induction and maintenance, Wt mice bearing the graft for 42 days were depleted of CD25⁻ T cells using an anti-CD25 antibody. 50% of mice (2/4) rejected the graft between day 14 and 20, suggesting a major role for T_{regs} and an involvement of additional mechanisms in this model (FIG. 6a). This was furthermore confirmed by transfer of CD4⁺CD25⁺ and CD4⁻ T cells of tolerant mice which accepted the graft for 98 days, as transfer of CD4⁻T cells lead to graft rejection. CD4⁺CD25⁺ T cells were able to inhibit graft rejection indicating further mechanisms enhancing and supporting the effects of T_{regs} (FIG. 6b).

### REFERENCES

Bala K K and Moudgil K D (2006) Induction and maintenance of self tolerance: the role of CD4+CD25+ regulatory T cells Arch Immunol Ther Exp (Warsz) 54:307-321;

Condon C, Watkins S C, Celluzzi C M, Thompson K, and Falo L D, Jr. (1996) DNA-based immunization by in vivo transfection of dendritic cells Nat Med 2:1122-1128;

Gandhi K, Chen M, Aasi S, Lapiere J C, Woodley D T, and Chan L S (2000) Autoantibodies to type VII collagen have heterogeneous subclass and light chain compositions and their complement-activating capacities do not correlate with the inflammatory clinical phenotype J Clin Immunol 20:416-423;

Ghazizadeh S, Kalish R S, and Taichman L B (2003) Immune-mediated loss of transgene expression in skin: Implications for cutaneous gene therapy Molecular Therapy 7:296-303;

Goudy K S, Wang B, and Tisch R (2008) Gene gun-mediated DNA vaccination enhances antigen-specific immunotherapy at a late preclinical stage of type 1 diabetes in nonobese diabetic mice Clin Immunol 129:49-57;

Holcmann M, Stoitzner P, Drobits B, Luehrs P, Stingl G, Romani N et al. (2009) Skin inflammation is not sufficient to break tolerance induced against a novel antigen J Immunol 183:1133-1143;

Joffre O, Santolaria T, Calise D, Al Saati T, Hudrisier D, Romagnoli P et al. (2008) Prevention of acute and chronic allograft rejection with CD4+CD25+Foxp3+ regulatory T lymphocytes Nat Med 14:88-92;

Kageyama Y, Koide Y, Uchijima M, Nagata T, Yoshida A, Taiki A et al. (2004) Plasmid encoding interleukin-4 in the amelioration of murine collagen-induced arthritis Arthritis Rheum 50:968-975;

Kataoka M, Shimizu Y, Margenthaler J A, Landeros K, Otomo N, and Flye M W (2002) Transfer of “infectious” cardiac allograft tolerance induced by donor-specific transfusion Surgery 132:167-172;

Laimer M, Bauer J W, Klausegger A, Koller J, Pohla-Gubo G, Muss W et al. (2006) Skin grafting as a therapeutic approach in pretibially restricted junctional epidermolysis bullosa Br J Dermatol 154:185-187;

Lobell A, Weissert R, Eltayeb S, de Graaf K L, Wefer J, Storch M K et al. (2003) Suppressive DNA vaccination in myelin oligodendrocyte glycoprotein peptide-induced experimental autoimmune encephalomyelitis involves a T1-biased immune response J Immunol 170:1806-1813;

Ludwig R J, Recke A, Bieber K, Muller S, Marques A D, Banczyk D et al. (2010) Generation of Antibodies of Distinct Subclasses and Specificity Is Linked to H2s in an Active Mouse Model of Epidermolysis Bullosa Acquisita J Invest Dermatol 2011 Jan;131(1):167-76. Epub 2010 Aug 19;

Mavilio F, Pellegrini G, Ferrari S, Di Nunzio F, Di Iorio E, Recchia A et al. (2006) Correction of junctional epidermolysis bullosa by transplantation of genetically modified epidermal stem cells Nat Med 12:1397-1402;

Murauer E M, Gache Y, Gratz I K, Klausegger A, Muss W, Gruber C et al. (2011) Functional correction of type VII collagen expression in dystrophic epidermolysis bullosa J Invest Dermatol 131:74-83;

Olasz E B, Roh J, Yee C L, Arita K, Akiyama M, Shimizu H et al. (2007) Human bullous pemphigoid antigen 2 transgenic skin elicits specific IgG in wild-type mice J Invest Dermatol 127:2807-2817;

Qin S, Cobbold S P, Pope H, Elliott J, Kioussis D, Davies J et al. (1993) “Infectious” transplantation tolerance Science 259:974-977;

Rosenberg A S and Singer A (1988) Evidence that the effector mechanism of skin allograft rejection is antigen-specific Proc Natl Acad Sci USA 85:7739-7742;

Ruiz P J, Garren H, Ruiz I U, Hirschberg D L, Nguyen L V, Karpuj M V et al. (1999) Suppressive immunization with DNA encoding a self-peptide prevents autoimmune disease: modulation of T cell costimulation J Immunol 162:3336-3341;

Sitaru C, Chiriac M T, Mihai S, Buning J, Gebert A, Ishiko A et al. (2006) Induction of complement-fixing autoantibodies against type VII collagen results in subepidermal blistering in mice Journal of Immunology 177:3461-3468;

Spirito F, Meneguzzi G, Danos O, and Mezzina M (2001) Cutaneous gene transfer and therapy: the present and the future Journal of Gene Medicine 3:21-31;

Stoecklinger A, Eticha T D, Mesdaghi M, Kissenpfennig A, Malissen B, Thalhamer J et al. (2011) Langerin+ Dermal Dendritic Cells Are Critical for CD8+ T Cell Activation and IgH {gamma}-1 Class Switching in Response to Gene Gun Vaccines J lmmunol 186:1377-1383;

Stoecklinger A, Grieshuber I, Scheiblhofer S, Weiss R, Ritter U, Kissenpfennig A et al. (2007) Epidermal langerhans cells are dispensable for humoral and cell-mediated immunity elicited by gene gun immunization J Immunol 179:886-893;

Tang D C, DeVit M, and Johnston S A (1992) Genetic immunization is a simple method for eliciting an immune response Nature 356:152-154;

von Herrath M G and Harrison L C (2003) Antigen-induced regulatory T cells in autoimmunity Nat Rev Immunol 3:223-232;

Wally V, Klausegger A, Koller U, Lochmuller H, Krause S, Wiche G et al. (2008) 5′ trans-splicing repair of the PLEC1 gene J Invest Dermatol 128:568-574.

1. A composition for use in the prevention of the rejection of skin tissue, comprising an effective amount of:
a) a peptide comprising an epitope of an antigen selected from type XVII collagen, type VII collagen, integrin alpha 6, integrin beta 4, chains of laminin, chains of laminin 322, type IV collagen, plectin, plakoglobin, bullous pemphigoid antigen 1, periplakin, envoplakin, desmoglein 1, desmoglein 3, a desmocollin and human bullous pemphigoid antigen 2 (hBPAG2), wherein said epitope induces immunological tolerance against its underlying polypeptide, and/or
b) a nucleic acid for expressing a peptide comprising an epitope of said antigen, and wherein said epitope is not the full length polypeptide.

2. The composition according to claim 1, wherein said epitope is located in a peptide comprising a extracellular domain of hBPAG2.

3. The composition according to claim 1, wherein said composition is suitable for gene therapy.

4. The composition according to claim 1, wherein said composition is suitable for gene gun transfer.

5. The composition according to claim 1, wherein said composition is formulated for topical application.

6. A method for the treatment of a genetic skin disease or an autoimmune blistering disease wherein said method comprises administering, to a subject in need of such treatment, a composition of claim 1.

7. The method according to claim 6 used for the treatment of bullous pemphigoid, epidermolysis bullosa (simplex, junctional, dystrophic) and/or pemphigus.

8. A method for the prevention of the rejection of skin tissue, comprising administering to a subject in need of such prevention an effective amount of a composition according to claim 1.

9. The method according to claim 8, wherein said prevention comprises gene therapy.

10. The method according to claim 8, wherein said composition is administered using gene gun transfer.

11. The method according to claim 8, wherein said composition is administered topically.

12. The method according to claim 6, used for the treatment of pemphigus vulgaris, paraneoplastic pemphigus, bullous pemphigoid, cicatricial pemphigoid, dermatitis herpetiformis, linear IgA dermatosis, and/or epidermolysis bullosa acquisita.

13. The composition, according to claim 1, wherein said epitope is NC16A.

14. The composition, according to claim 3, wherein said gene therapy is corrective gene therapy and/or gene replacement therapy.

15. The composition, according to claim 5, formulated as a gel, creme, paste, lotion, spray, suspension, dispersion salve, hydrogel, ointment formulation or for gene gun transfer.

16. The method, according to claim 9, wherein said gene therapy is corrective gene therapy and/or gene replacement therapy.

17. The method, according to claim 11, wherein said composition is administered via a gel, creme, paste, lotion, spray, suspension, dispersion salve, hydrogel, ointment formulation or via gene gun transfer.
